(19) United States
(12) Patent Application Publication
(10) Pub. No.: US 2007/0249909 A1
(43) Pub. Date: Oct. 25, 2007

(54) Catheter configurations

(76) Inventors: Angela Kornkven Volk, Rogers, MN (US); John Blix, Maple Grove, MN (US); Richard Olson, Blaine, MN (US)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 11/411,690

(22) Filed: Apr. 25, 2006

(51) Int. Cl. A61B 1/00

(52) U.S. Cl. 600/184

(57) ABSTRACT

The present invention is directed to variations of catheter configurations, wherein the outer shafts have been supplemented with electroactive polymer (EAP) material to modify the performance characteristics of the catheter.

### FIELD OF THE INVENTION

This invention relates to an assembly and method for delivering and deploying an expandable medical device, particularly within a lumen of a body vessel. More specifically, this invention relates to the application of electroactive polymers (EAP) on catheter assemblies.

### BACKGROUND OF THE INVENTION

Percutaneous transluminal coronary angioplasty (PTCA) is a procedure that is well established for the treatment of blockages, lesions, stenosis, thrombus, etc. present in body lumens such as the coronary arteries and/or other vessels.

A widely used form of percutaneous coronary angioplasty makes use of a dilatation balloon catheter, which is introduced into and advanced, through a lumen or body vessel until the distal end thereof is at a desired location in the vasculature. Once in position across an afflicted site, the expandable portion of the catheter, or balloon, is inflated to a predetermined size with a fluid at relatively high pressures. By doing so the vessel is dilated, thereby radially compressing the atherosclerotic plaque of any lesion present against the inside of the artery wall, and/or otherwise treating the afflicted area of the vessel. The balloon is then deflated to a small profile so that the dilatation catheter may be withdrawn from the patient's vasculature and blood flow resumed through the dilated artery.

In angioplasty procedures of the kind described above, there may be restenosis of the artery, which either necessitates another angioplasty procedure, a surgical by-pass operation, or some method of repairing or strengthening the area. To reduce restenosis and strengthen the area, a physician can implant an intravascular prosthesis for maintaining vascular patency, such as a stent, inside the artery at the lesion.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The present invention is directed to variations of catheter configurations, wherein the outer shafts or sheaths include an electroactive polymer (EAP) material to modify the performance characteristics of the catheter.

In at least one embodiment a catheter is provided for use in a body lumen, the catheter includes at least one active region. The at least one active region is at least partially formed of electroactive polymer material.

In at least one embodiment, a retractable sheath of a catheter is supplemented with EAP material to provide active regions comprising electroactive polymer material. When activated, the EAP material radially expands the distal sheath to reduce deployment forces when it is retracted from over the stent. The EAP material is oriented in a pattern such that when the EAP material expands, it increases the diameter of the distal sheath to lessen the friction between the distal sheath and the loaded stent.

In at least one embodiment, a retraction sheath of a catheter is supplemented with EAP material to provide active regions comprising electroactive polymer material. When activated, the EAP material longitudinally contracts or shortens the retraction sheath to withdraw a distal sheath from over the loaded stent.

In at least one embodiment, the proximal end of a distal sheath including EAP is fixed to allow for the longitudinal shortening of the distal sheath. The EAP material is oriented in a pattern such that when the EAP material is activated, it decreases the length of the distal sheath, withdrawing it from over the loaded stent.

In at least one embodiment, the proximal end of a retraction sheath including EAP is fixed to allow for the longitudinal shortening of the retraction sheath. The EAP material is oriented in a pattern such that when the EAP material is activated, it decreases the length of the retraction sheath to withdraw the distal sheath and release the stent.

In at least one embodiment, a catheter is outfitted with spiral fan blade shaped elements positioned on the outer surface of the catheter at positions along its length. The fan blade elements are supplemented with EAP material to extend radially for blood movement.

In some embodiments, the EAP may be formed from an anionic electroactive polymer.

In at least one embodiment, the EAP is electrically engaged and is in electrical communication with a source of anions.

In certain other embodiments, the medical devices of the present invention are actuated, at least in part, using materials involving piezoelectric, electrostrictive, and/or Maxwell stresses.

In at least one embodiment, a catheter is outfitted with fan blade shaped elements positioned on the outer surface of the catheter at positions along its length. The fan blade elements include EAP material to extend radially for blood movement.

In at least on embodiment, the outer shaft of a catheter is supplemented with EAP to provide contraction of the midshaft bond and distal shaft for a use in kissing balloon technique, such as described in U.S. Publication 2005/0102023A1.

In the embodiments discussed, the EAP material may be applied to the inner or outer diameter of the sheaths or it may be incorporated into the material of the sheaths material.

In the embodiments discussed, the supplemented components of the catheter discussed may be combined and mixed for uniform dispersion within the EAP material. Following mixing, EAP material may be extruded into the desired form.

These and other embodiments which characterize the invention are pointed out with particularity in the claims annexed hereto and forming a part hereof. The drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1A shows an electroactive polymer in a first state having a length dimension and a second state having a different length dimension.

FIG. 1B shows an alternative electroactive polymer in a first arcuate state and a second arcuate state.

FIG. 1C shows an alternative electroactive polymer in a first state having a first volume and a second state having a different second volume.

FIG. 2 shows a side view of a catheter according to an alternative embodiment of the invention having a loaded stent including a cross-sectional view of the distal portion thereof and a side view of the proximal end of a catheter according to the invention showing the manifold portion thereof.

FIG. 3 shows a side view of a catheter according to an alternative embodiment of the invention having a loaded stent including a cross-sectional view of the distal portion thereof, wherein the loaded stent is shown as partially deployed, and a side view of the proximal end of a catheter according to the invention showing the manifold portion thereof.

FIG. 4 shows a side view of a catheter according to an alternative embodiment of the invention having a loaded stent including a cross-sectional view of the distal portion thereof, wherein the loaded stent is shown as fully deployed and a side view of the proximal end of a catheter according to the invention showing the manifold portion thereof.

FIG. 5 shows a side view of a catheter according to an alternative embodiment of the invention having a loaded stent including a cross-sectional view of the distal portion thereof.

FIG. 6 shows a side view of a catheter according to an alternative embodiment of the invention having a loaded stent including a cross-sectional view of the distal portion thereof, wherein the loaded stent is shown as fully deployed.

FIG. 7 shows a side view of a catheter according to an alternative embodiment of the invention having a loaded stent including a cross-sectional view of the distal portion thereof.

FIG. 8 is a sectional view of the catheter thereof, taken along line 8-8 in FIG. 7.

FIG. 9 shows a side view of a catheter according to an alternative embodiment of the invention having a loaded stent including a cross-sectional view of the distal portion thereof.

FIGS. 10A-B show partial cross-sectional side views of an alternative embodiment of the invention.

FIGS. 11A-B show partial cross-sectional side views of an alternative embodiment of the invention.

FIG. 12 shows a partial cross-sectional side view of an alternative embodiment of the invention.

FIGS. 13A-B show partial side views of an alternative embodiment of the invention.

FIG. 14A shows a partial cross-sectional side view of an alternative embodiment of the invention.

FIG. 14B shows a partial perspective view of a portion of the embodiment shown in FIG. 14A.

FIG. 14C shows a partial cross-sectional side view of the alternative embodiment of the invention shown in FIG. 14A when activated.

FIGS. 15A-B show partial side views of an alternative embodiment of the invention.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

Depicted in the figures are various aspects of the invention. Elements depicted in one figure may be combined with, or substituted for, elements depicted in another figure as desired.

The present invention relates to strategic placement or use of electroactive polymers (EAP). Depending on the placement of EAP, a variety of characteristics may be manipulated and/or improved. Particular portions of the catheter configurations of the present invention may be actuated, at least in part, with electroactive polymer (EAP) actuators. Electroactive polymers are characterized by their ability to change shape in response to electrical stimulation. EAPs include electric EAPs and ionic EAPs. Piezoelectric materials may also be employed but tend to undergo deformation when voltage is applied.

Electric EAPs include ferroelectric polymers, dielectric EAPs, electrorestrictive polymers such as the electrorestrictive graft elastomers and electro-viscoelastic elastomers, and liquid crystal elastomer materials.

Ionic EAPs include ionic polymer gels, ionomeric polymer-metal composites, conductive polymers and carbon nanotubes. Upon application of a small voltage, ionic EAPs may bend significantly. Ionic EAPs also have a number of additional properties that make them attractive for use in the devices of the present invention, including the following: (a) they are lightweight, flexible, small and easily manufactured; (b) energy sources are available which are easy to control, and energy may be easily delivered to the EAPS; (c) small changes in potential (e.g., potential changes on the order of 1V) may be used to effect volume change in the EAPs; (d) they are relatively fast in actuation (e.g., full expansion/contraction in a few seconds); (e) EAP regions may be created using a variety of techniques, for example, electrodeposition; and (f) EAP regions may be patterned, for example, using photolithography, if desired.

Conductive plastics may also be employed. Conductive plastics include common polymer materials which are almost exclusively thermoplastics that require the addition of conductive fillers such as powdered metals or carbon (usually carbon black or fiber).

Ionic polymer gels are activated by chemical reactions and may become swollen upon a change from an acid to an alkaline environment.

Monomeric polymer-metal composites may bend as a result of the mobility of cations in the polymer network. Suitable base polymers include perfluorosulfonate and perfluorocarboxylate.

Essentially any electroactive polymer that exhibits contractile or expansile properties may be used in connection with the various active regions of the invention, including any of those listed above.

In some embodiments herein, the EAPs employed are ionic EAPs, more specifically, the ionic EAPs are conductive polymers that feature a conjugated backbone (they include a backbone that has an alternating series of single and double carbon-carbon bonds, and sometimes carbon-nitrogen bonds, i.e. π-conjugation) and have the ability to increase the electrical conductivity under oxidation or reduction. Such polymers allow freedom of movement of electrons, therefore allowing the polymers to become conductive. The pi-conjugated polymers are converted into electrically conducting materials by oxidation (p-doping) or reduction (n-doping).

The volume of these polymers changes dramatically through redox reactions at corresponding electrodes through exchanges of ions with an electrolyte. The EAP-containing active region contracts and expands in response to the flow of ions out of, or into, the same. These exchanges occur with small applied voltages and voltage variation may be used to control actuation speeds.

Any of a variety of pi-conjugated polymers may be employed herein. Examples of suitable conductive polymers include, but are not limited to, polypyrroles, polyanilines, polythiophenes, polyethylenedioxythiophenes, poly(p-phenylenes), poly(p-phenylene vinylene)s, polysulfones, polypyridines, polyquinoxalines, polyanthraquinones, poly(N-vinylcarbazole)s and polyacetylenes, with the most common being polythiophenes, polyanilines, and polypyrroles.

Some of the structures are shown below:

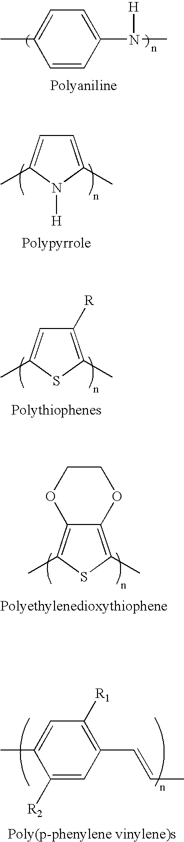

Polypyrrole, shown in more detail below, is one of the most stable of these polymers under physiological conditions:

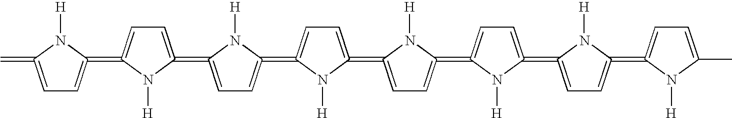

The above list is intended for illustrative purposes only, and not as a limitation on the scope of the present invention.

The behavior of conjugated polymers is dramatically altered with the addition of charge transfer agents (dopants). These materials may be oxidized to a p-type doped material by doping with an anionic dopant species or reducible to a n-type doped material by doping with a cationic dopant species. Generally, polymers such as polypyrrole (PPy) are partially oxidized to produce p-doped materials:

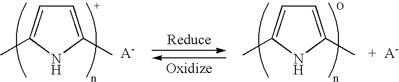

Dopants have an effect on this oxidation-reduction scenario and convert semi-conducting polymers to conducting versions close to metallic conductivity in many instances. Such oxidation and reduction are believed to lead to a charge imbalance that, in turn, results in a flow of ions into or out of the material. These ions typically enter/exit the material from/into an ionically conductive electrolyte medium associated with the electroactive polymer.

Dimensional or volumetric changes may be effectuated in certain polymers by the mass transfer of ions into or out of the polymer. This ion transfer is used to build conductive polymer actuators (volume change). For example, in some conductive polymers, expansion is believed to be due to ion insertion between chains, whereas in others inter-chain repulsion is believed to be the dominant effect. Regardless of the mechanism, the mass transfer of ions into and out of the material leads to an expansion or contraction of the polymer, delivering significant stresses (e.g., on the order of 1 MPa) and strains (e.g., on the order of 10%). These characteristics are ideal for construction of the devices of the present invention. As used herein, the expansion or the contraction of the active region of the device is generally referred to as “actuation.”

The following elements are commonly utilized to bring about electroactive polymer actuation: (a) a source of electrical potential, (b) an active region, which comprises the electroactive polymer, (c) a counter electrode and (d) an electrolyte in contact with both the active region and the counter electrode.

The source of electrical potential for use in connection with the present invention may be quite simple, consisting, for example, of a dc battery and an on/off switch. Alternatively, more complex systems may be utilized. For example, an electrical link may be established with a microprocessor, allowing a complex set of control signals to be sent to the EAP-containing active region(s).

The electrolyte, which is in contact with at least a portion of the surface of the active region, allows for the flow of ions and thus acts as a source/sink for the ions. Any suitable electrolyte may be employed herein. The electrolyte may be, for example, a liquid, a gel, or a solid, so long as ion movement is permitted. Examples of suitable liquid electrolytes include, but are not limited to, an aqueous solution containing a salt, for example, an NaCl solution, a KCl solution, a sodium dodecylbenzene sulfonate solution, a phosphate buffered solution, physiological fluid, etc. Examples of suitable gel electrolytes include, but are not limited to, a salt-containing agar gel or polymethylmethacrylate (PMMA) gel. Solid electrolytes include ionic polymers different from the EAP and salt films.

The counter electrode may be formed from any suitable electrical conductor, for example, a conducting polymer, a conducting gel, or a metal, such as stainless steel, gold or platinum. At least a portion of the surface of the counter electrode is generally in contact with the electrolyte, in order to provide a return path for charge.

In one specific embodiment, the EAP employed is polypyrrole. Polypyrrole-containing active regions may be fabricated using a number of known techniques, for example, extrusion, casting, dip coating, spin coating, or electro-polymerization/deposition techniques. Such active regions may also be patterned, for example, using lithographic techniques, if desired.

As a specific example of a fabrication technique, polypyrrole may be galvanostatically deposited on a platinised substrate from a pyrrole monomer solution using the procedures described in D. Zhou et al., “Actuators for the Cochlear Implant,” Synthetic Metals 135-136 (2003) 39-40. Polypyrrole may also be deposited on gold. In some embodiments, adhesion of the electrodeposited polypyrrole layer is enhanced by covering a metal such as gold with a chemisorbed layer of molecules that may be copolymerized into the polymer layer with chemical bonding. Thiol is one example of a head group for strong chemisorbtion to metal. The tail group may be chemically similar to structured groups formed in the specific EAP employed. The use of a pyrrole ring attached to a thiol group (e.g., via a short alkyl chain) is an example for a polypyrrole EAP. Specific examples of such molecules are 1-(2-thioethyl)-pyrrole and 3-(2-thioethyl)-pyrrole. See, e.g., E. Smela et al., “Thiol Modified Pyrrole Monomers: 1. Synthesis, Characterization, and Polymerization of 1-(2-Thioethyl)-Pyrrole and 3-(2-Thioethyl)-Pyrrole,” Langmuir, 14 (11), 2970-2975, 1998.

Various dopants may be used in the polypyrrole-containing active regions, including large immobile anions and large immobile cations. According to one specific embodiment, the active region comprises polypyrrole (PPy) doped with dodecylbenzene sulfonate (DBS) anions. When placed in contact with an electrolyte containing small mobile cations, for example, Na⁺ cations, and when a current is passed between the polypyrrole-containing active region and a counter electrode, the cations are inserted/removed upon reduction/oxidation of the polymer, leading to expansion/contraction of the same. This process may be represented by the following equation:
PPy⁺(DBS⁻)+Na⁺+e⁻ [symbol] PPy^{o}(Na⁺DBS⁻)
where Na⁺ represents a sodium ion, e⁻ represents an electron, PPy⁺ represents the oxidized state of the polypyrrole, PPy^{o} represents the reduced state of the polymer, and species are enclosed in parentheses to indicate that they are incorporated into the polymer. In this case the sodium ions are supplied by the electrolyte that is in contact with the electroactive polymer member. Specifically, when the EAP is oxidized, the positive charges on the backbone are at least partially compensated by the DBS⁻ anions present within the polymer. Upon reduction of the polymer, however, the immobile DBS⁻ ions cannot exit the polymer to maintain charge neutrality, so the smaller, more mobile, Na⁺ ions enter the polymer, expanding the volume of the same. Upon re-oxidation, the Na⁺ ions again exit the polymer into the electrolyte, reducing the volume of the polymer.

EAP-containing active regions may be provided that either expand or contract when an applied voltage of appropriate value is interrupted depending, for example, upon the selection of the EAP, dopant, and electrolyte.

Additional information regarding EAP actuators, their design considerations, and the materials and components that may be employed therein, may be found, for example, in E. W. H. Jager, E. Smela, O. Inganäs, “Microfabricating Conjugated Polymer Actuators,” Science, 290, 1540-1545, 2000; E. Smela, M. Kallenbach, and J. Holdenried, “Electrochemically Driven Polypyrrole Bilayers for Moving and Positioning Bulk Micromachined Silicon Plates,” J. Microelectromechanical Systems, 8(4), 373-383, 1999; U.S. Pat. No. 6,249,076, assigned to Massachusetts Institute of Technology, and Proceedings of the SPIE, Vol. 4329 (2001) entitled “Smart Structures and Materials 2001: Electroactive Polymer and Actuator Devices (see, e.g., Madden et al, “Polypyrrole actuators: modeling and performance,” at pp. 72-83), each of which is hereby incorporated by reference in its entirety.

Furthermore, networks of conductive polymers may also be employed. For example, it has been known to polymerize pyrrole in electroactive polymer networks such as poly(vinylchloride), poly(vinyl alcohol), NAFION®, a perfluorinated polymer that contains small proportions of sulfonic or carboxylic ionic functional groups, available from E.I. DuPont Co., Inc. of Wilmington, Del.

Electroactive polymers are also discussed in detail in commonly assigned copending U.S. patent application Ser. No. 10/763,825, the entire content of which is incorporated by reference herein. Further information regarding EAP may be found in U.S. Pat. No. 6,514,237, the entire content of which is incorporated by reference herein.

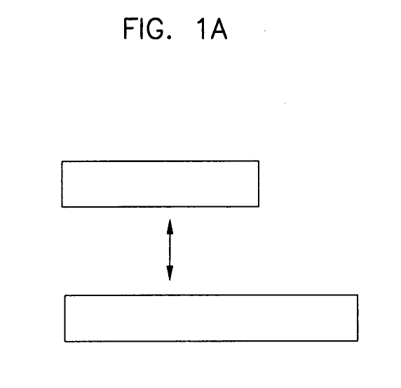

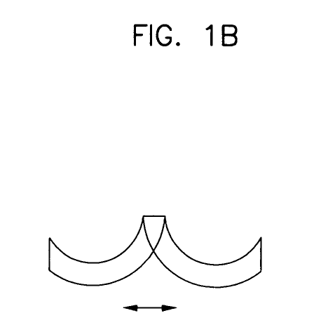

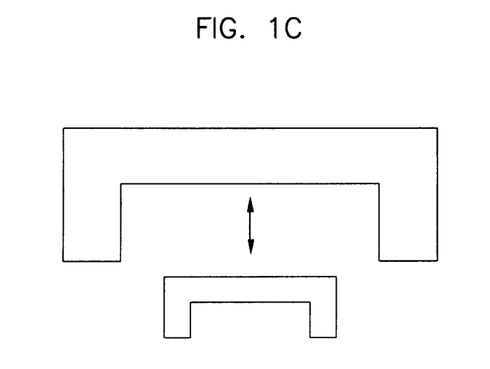

Turning now to the figures, as depicted in FIG. 1A, the exposure of anions to the EAP material may cause expansion and contraction in a longitudinal dimension. Alternatively, as depicted in FIG. 1B the exposure of anions to the EAP material may cause a change in the arcuate direction or orientation of the material. The radius of the arcuate curvature may be as small as a few μm. As depicted in FIG. 1C the exposure of anions to the EAP material may cause the volume and/or length, width, and height dimension of the EAP material to enlarge.

The extent of the expansion of the EAP material in either a length and/or width dimension, following exposure to anions, may vary between a few μm to several centimeters. Generally, the thickness dimensions are selected as needed for the application. For example, in some embodiments, dimensions are selected are between 0.0005 to 0.010 inches. The speed of the EAP material for expansion or contraction may be selected for the particular application. In some embodiments, the speed of the expansion or contraction of the material may vary between less than 0.5 seconds to approximately 10 seconds per cycle. The speed of the EAP expansion or contraction is generally dependent upon the thickness dimension selected. Thinner EAP materials expand and/or contract at an increased rate as compared to thicker EAP materials.

Generally a voltage of −1.5 to 1.5 volts is utilized to provide the desired anions or cations for implementation of a state change for the EAP into either a pre-delivery or delivery state. For some EAP's a voltage range of −5 to 5 volts is needed to provide the desired change.

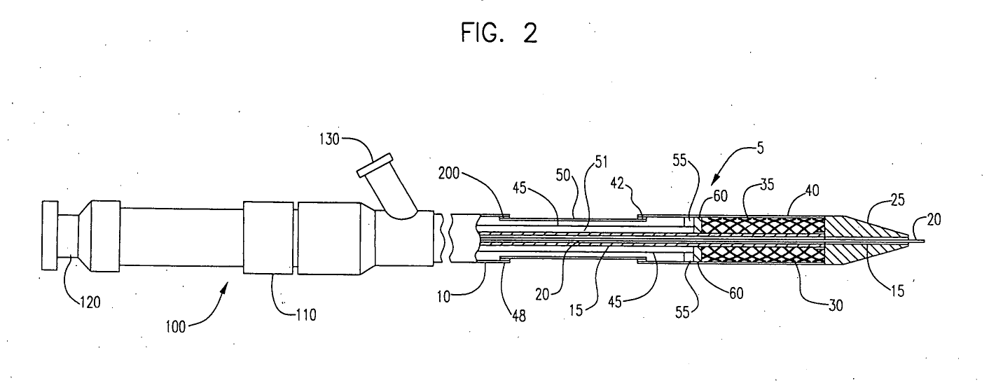

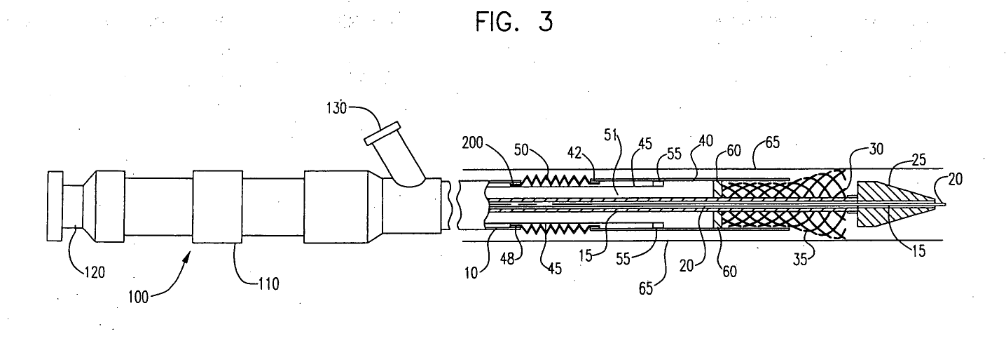

FIGS. 2-4 illustrate three stages of the deployment of a self-expanding stent 35 using the shown embodiment of the catheter of the present invention FIG. 2 represents a loaded deployment catheter 5 with the stent 35 covered by the distal sheath/shaft 40 and the retraction sheath 50 in its extended state. The retraction sheath 50 is also considered to be a midshaft. FIG. 3 shows the stent 35 partially deployed, with the distal sheath retracted to cause the retraction sheath 50 to partially collapse. In some embodiments, as mentioned above, the retraction sheath 50 is electronically actuated causing the distal sheath 40 to be pulled back. The stent is prevented from moving proximally with the distal sheath 40 by the stopper and therefore, the stent 35 begins to release and expand while the retraction sheath 50 begins to collapse upon itself.

FIG. 2 shows a cross-section of the distal portion of an embodiment of a stent delivery catheter, generally designated as 5. The device generally comprises a proximal outer 10 which covers the majority of the catheter 5 excluding a portion of the distal end of the catheter 5. The proximal outer 10 encloses an optional guide wire shaft 15 which extends through and terminates with the distal tip 25 of the catheter 5. The guide wire shaft 15 encloses a guide wire 20 which aids in the navigation of the catheter 5 through the appropriate vessel.

Situated just proximal to the distal tip 25 is the portion 30 of catheter 5 around which the stent is concentrically carried. The stent 35 surrounds the guide wire shaft 15. The stent may be a self-expanding stent or a balloon expandable stent carried by an expansion balloon. Self-expanding and balloon expandable stents are well known in the art and require no further instruction.

The embodiment shown further comprises a retractable distal sheath 40 which covers and contains the loaded stent 35. The retractable distal sheath 40 covers the stent 35 in its reduced delivery configuration. In the case of a balloon catheter, the balloon would be positioned within the stent 35.

In at least one embodiment, the retractable distal sheath 40 is supplemented with EAP material to provide active regions comprising electroactive polymer material. When activated, the EAP material radially expands the distal sheath 40 to reduce deployment forces when it is retracted from over the stent. The EAP material is oriented in a pattern such that when the EAP material expands, it increases the diameter of the distal sheath 40 to lessen the friction between the distal sheath 40 and the stent 35. The EAP material may be applied to the inner or outer diameter of the distal sheath 40 or it may be incorporated into the material of the distal sheath 40.

Current can be supplied through wires extending to the EAP. The electrical supply can be either from a portable unit, such as a battery, or supplied from an AC source. The current may be controlled via a simple switch or a controller, such as an integrated circuit.

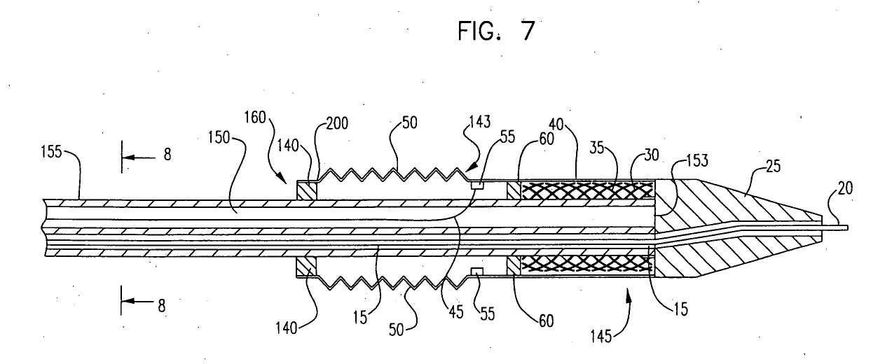

The distal sheath 40 is connected to a electrical lead 45, which allows a physician to electronically communicate with the EAP supplemented retractable sheath 40 to retract the distal sheath 40 from the proximal end of the catheter 5, thus releasing the stent 35 in the targeted area of the vessel. In one embodiment, an electrical lead lumen 51 (also item 150 in FIG. 7) extends longitudinally under the proximal outer 10, and houses the electrical lead 45. The electrical lead lumen 51, 150, that houses the electrical lead 45 may also carry fluid for purging air from the catheter 5. The proximal end of the electrical lead 45 is connected to an electrical supply so as to allow the user the ability to apply current to the retractable sheath 40.

In the embodiments discussed herein, the distal sheath 40 may be combined and mixed for uniform dispersion within the EAP material. Following mixing, EAP material may be extruded into sheath form.

The embodiments additionally may comprise a retraction sheath 50 situated between the proximal outer 10 and the distal sheath 40. The retraction sheath 50 covers the exposed area between the proximal outer 10 and the distal sheath 40, serving to protect the guide wire shaft 15 and the electrical lead 45 in this area. The retraction sheath 50 is adhered to the proximal end of the distal sheath 40 at point 42 and the distal end of the proximal outer 10 at point 48. As the distal sheath 40 is retracted, the retraction sheath 50 is forced back, collapsing upon itself into an accordion type configuration to give the distal sheath 40 room to retract. The distal sheath 40 and the retraction sheath 50 may be two separate sheaths adhered to one another, or they may form one continuous sheath.

In at least one embodiment, the retraction sheath 50 is, along with or instead of the distal sheath 40, supplemented with EAP material to provide active regions comprising electroactive polymer material. An electrical lead, similar to that of electrical lead 45, may be utilized to activate the EAP material from the manifold 100. The EAP material transitions from a pre-deployment state and shortens to a post-deployment state. When activated, the EAP material longitudinally contracts or shortens the retraction sheath 50 to withdraw the distal sheath 40 from over the stent. Due to the addition of EAP material, the retraction sheath 50 does not have to be imparted with or an accordion shape and may, in fact, be a portion of the proximal outer 10 imparted with the EAP material, wherein the proximal outer 10 is directly connected to the distal sheath 40.

As can be seen in the illustrated embodiments, the proximal end 200 of the retraction sheath 50 is fixed relative to the guide wire shaft 15 to allow for the longitudinal shortening of the retraction sheath 50. The EAP material is oriented in a pattern such that when the EAP material is activated, it decreases the length of the retraction sheath 50 to withdraw the distal sheath 40 and release the stent 35. As mentioned above, the EAP material may be applied to the inner or outer diameter of the retraction sheath 50 or it may be incorporated into the material of the retraction sheath 50.

The distal sheath 40 may be connected via a collar comprised of a short section of hypotube 55, configured as an annular ring, to the electrical lead 45. The proximal end of the distal sheath 40 is attached to the annular ring 55 and the distal end of the electrical lead 45 is connected to the inside of the annular ring 55.

Proximal to the stent 35 is a stopper 60. The stopper 60 is attached to the guide wire shaft 15, or whatever may comprise the rigid inner core, and is used to prevent the stent 35 from moving proximally when the distal sheath 40 is retracted.

The proximal portion of the catheter 5, as shown in FIGS. 2-4, comprises of a manifold system, generally designated 100, which includes an electrical switch 110 connected to the electrical lead 45 and a power source (not shown). By actuating the switch 110, the distal sheath 40 and/or the retraction sheath 50 are/is retracted exposing the stent 35. The manifold 100 may further comprise a hydrating luer 130, which is preferably located on the distal end of the manifold 100 and is used to purge air from the catheter.

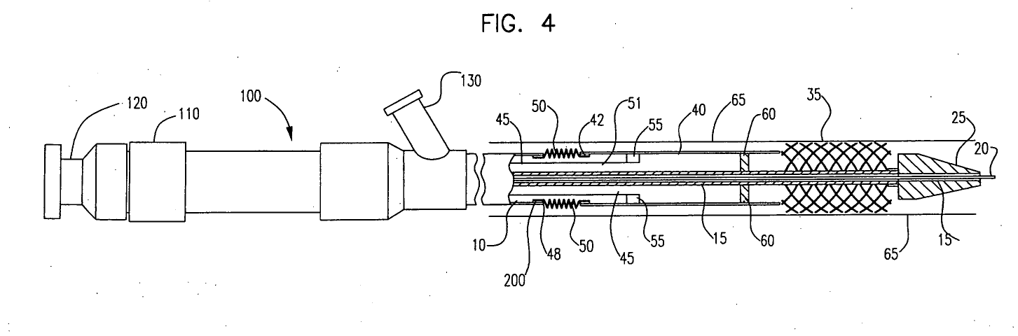

FIG. 4 shows the stent fully released. At this point the distal sheath 40 is fully retracted and the retraction sheath 50 is compressed releasing the stent 35 to allow it to self-expand against the vessel wall 65. After the stent 35 is expanded, the catheter 5 is withdrawn. It should be understood that a balloon expandable stent could also be utilized by arranging the stent around an optional placement balloon (not shown). Examples of balloon catheters may be found in U.S. 5968069 and U.S. U.S. Pat. No. 6,478,814. Once the sheath 40 is fully retracted the placement balloon would be inflated through its inflation lumen (not shown) to deploy the stent 35.

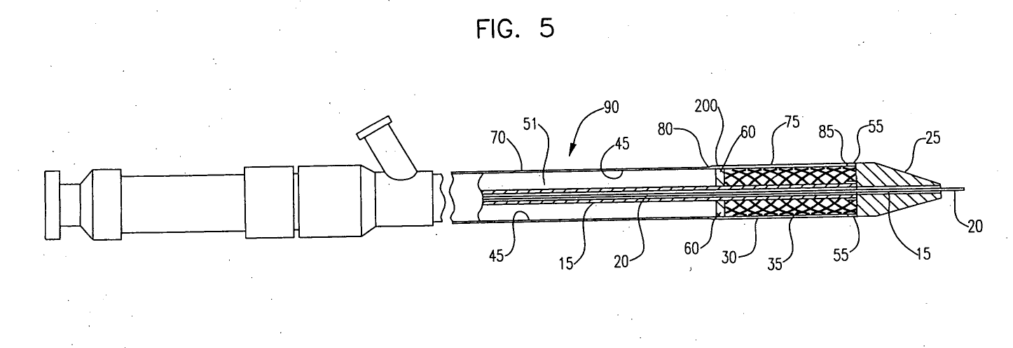

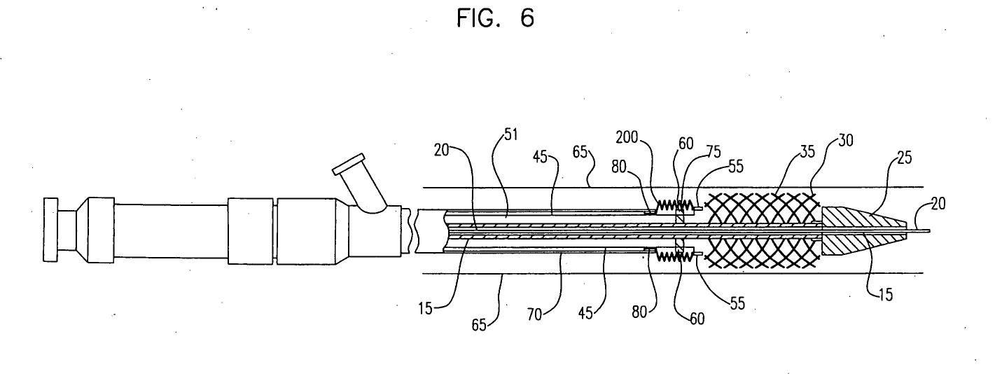

FIGS. 5 and 6 illustrate an alternative embodiment of the present invention. In this case, the proximal outer 70 extends distally over the catheter, generally designated 90, up to a position in close proximity with the stopper 60. Retraction sheath 75 performs as the distal sheath. The distal end of the proximal outer 70 is connected to the proximal end of the retraction sheath 75 at point 80. In this embodiment the collar 55 is connected to retraction sheath 75, which includes EAP material, at the distal end at point 85. As the electrical lead 45 is imparted with a current, the retraction sheath 75 is activated and drawn proximally and is retracted to release the stent 35. As discussed earlier, stopper 60 prevents the stent from moving proximally with the retracting sheath 75. FIG. 6 illustrates the fully retracted retraction sheath 75 and the release of the stent 35 to its fully expanded position urging against the inner wall of the vessel 65.

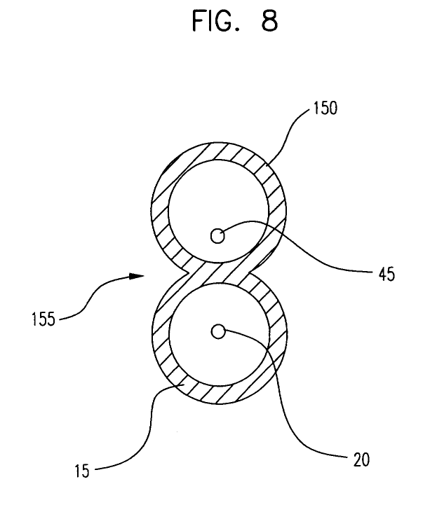

FIG. 7 discloses an alternative embodiment of the present invention. In this case the stent delivery system is generally designated 145 and the catheter 155 is comprised of a guide wire shaft 15 and an electrical lead lumen 150. The electrical lead lumen 150 is axially connected to the guide wire shaft 15, travelling along the length of the guide wire shaft 15 up to the distal tip 25 at point 153, as the guide wire shaft 15 continues through the distal tip 25. FIG. 8 illustrates the configuration of the catheter 155 from a cross-section perspective along lines 8-8 in FIG. 7. A stent 35 may be concentrically arranged around the catheter 15 near the distal end on the stent receiving portion 30. The device further comprises a retractable distal sheath 40 surrounding at least a portion of the stent 35.

FIG. 7 shows the retractable distal sheath 40 partly retracted. The proximal end of the retractable distal sheath 40 is attached to the retraction sheath 50 at point 143. The retraction sheath 50 is concentrically arranged around the catheter 155 and is shown in FIG. 7 as partially collapsed. The proximal end of the retraction sheath 50 is connected to a fixed anchoring device 140, such as an annular collar, which is affixed to the catheter 155 at point 160. The fixed anchoring device 140 stabilizes the proximal end of the retraction sheath 50 allowing it to collapse upon itself during retraction of the distal sheath 40.

The electrical lead 45 travels, proximal to distal, through the electrical lead lumen 150 and exits through an axial slit (not shown) in the surface of the electrical lead lumen 150. The distal end of the electrical lead 45 is attached to either the distal sheath 40 or the retraction sheath 50 or both. As mentioned above, either the retraction sheath 50 or the distal sheath 40 or both is/are imparted with EAP material. During the application of the device, current is applied through the electrical lead 45 to either the retraction sheath 50 and/or the distal sheath 40 resulting in the shortening of the either the retraction sheath 50 or the distal sheath 40 or both, thus freeing the stent 35 for delivery. The stopper 60 prevents the stent from moving proximally with the retracting sheath 75.

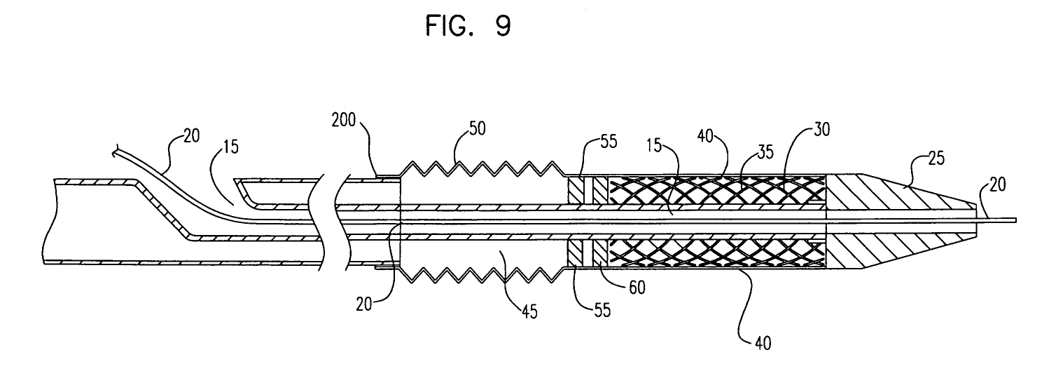

FIG. 9 illustrates a rapid exchange embodiment of the invention. The distal end of the catheter is structured and functions in the same fashion as that of the device shown in FIG. 2.

It should also be understood that the distal sheath 40 and the retraction sheath 50 may comprise one continuous sheath. It should also be understood that references and comments retraction sheath 50 may also be applied to retraction sheath 75.

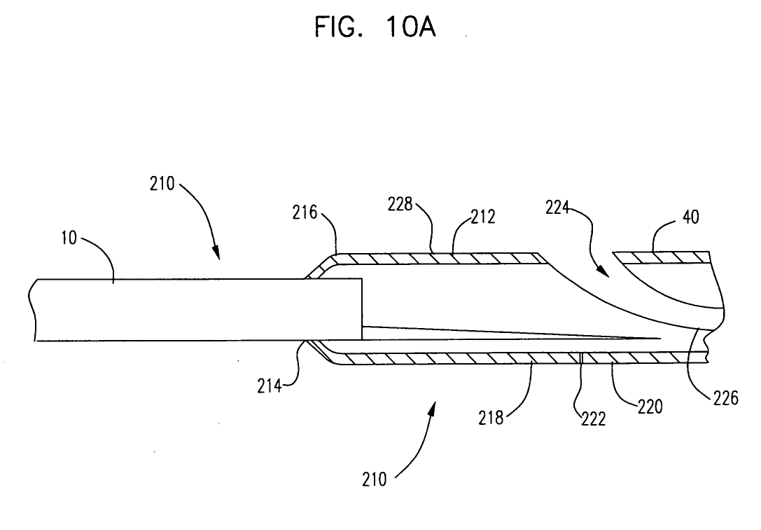

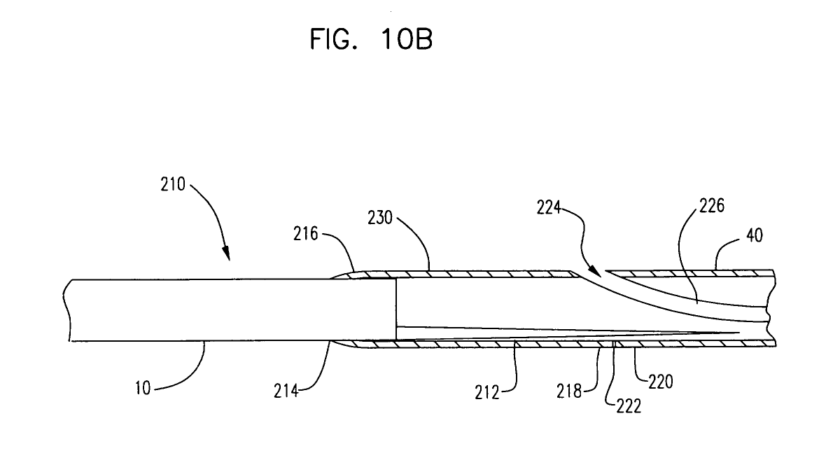

In at least one embodiment, as shown in FIGS. 10A and 100B, which shows a portion of a rapid exchange catheter 210, the proximal outer 10 is connected to the distal outer sheath/shaft 40 via a midshaft component 212. The proximal end 216 of the midshaft component 212 is connected to the distal end 214 end of the proximal outer 10 and the distal end 218 of the midshaft component 212 is connected to the proximal end 220 of the distal outer sheath/shaft 40 at a port bond 222. The components may be connected via suitable means such as, but not limited to, adhesion, welding, etc. In the particular embodiment shown, a port 224 is provided for access to a guide wire shaft 226.

The midshaft component 212 and/or distal outer shaft 40 may include EAP material. Upon activation of the EAP, the midshaft 212 and/or distal outer shaft 40 contracts from a first diameter 228, as shown in FIG. 10A, to a smaller diameter 230, as shown in FIG. 10B, resulting in a lower midshaft and/or port bond profile.

The EAP configuration in the particular embodiments can be of various configurations. The EAP material may be located on the outer surface, on the inner surface, inside the component or the entire wall thickness of the component.

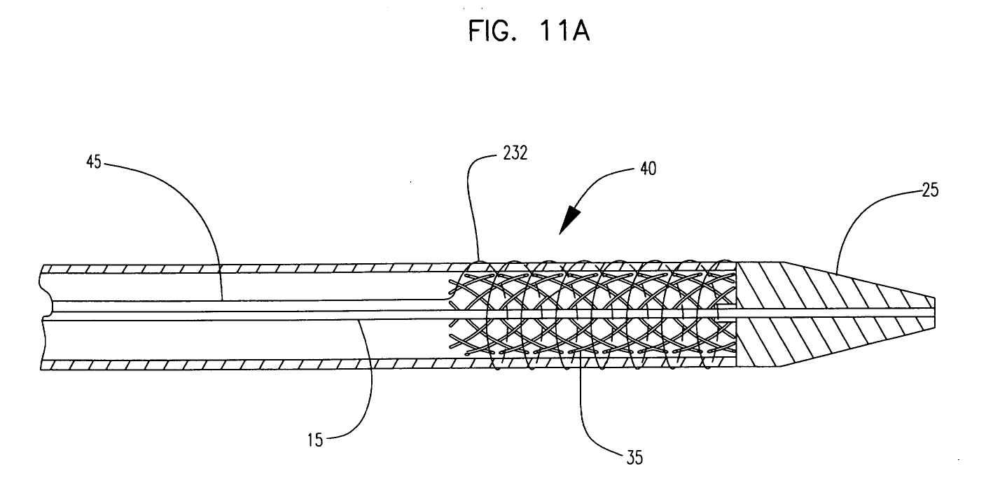

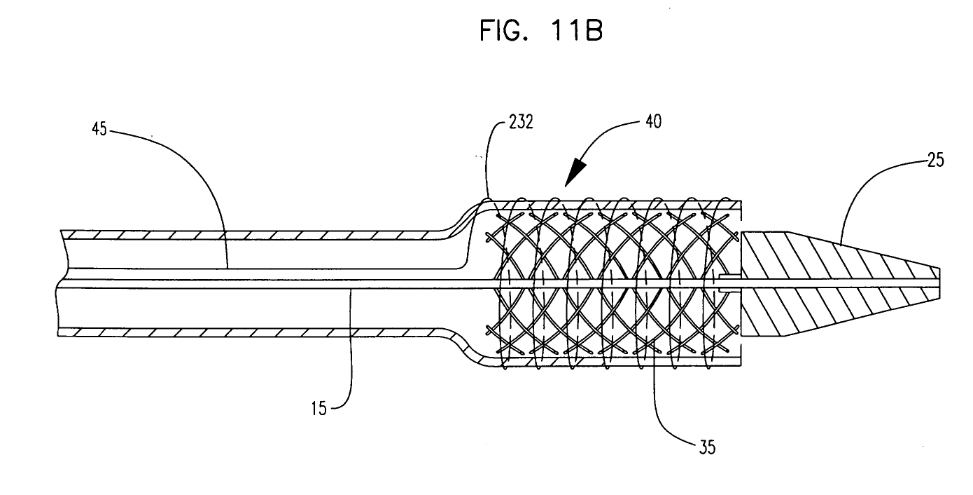

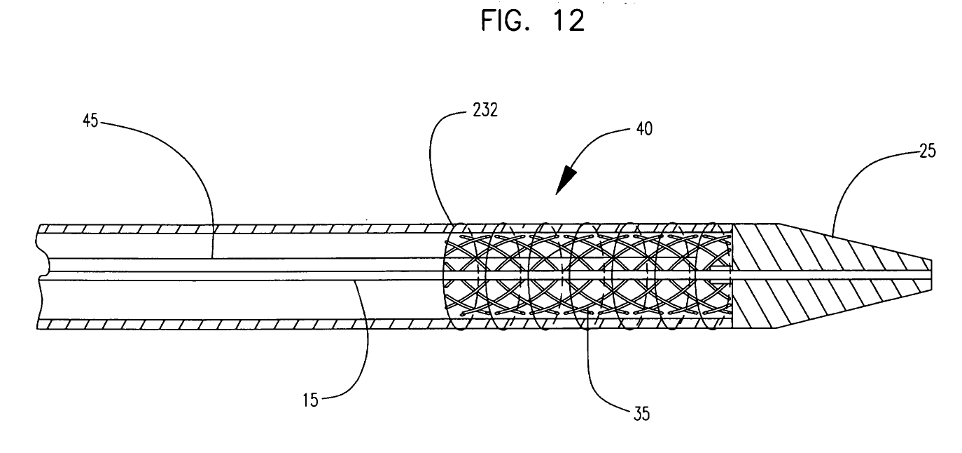

By way of example, as shown in FIGS. 11A-11B and 12, the EAP material 232 may be in a spiral shape, as shown in FIGS. 11A-11B, or circumferential rings, as shown in FIG. 12. In the particular embodiment shown in FIGS. 11A-11B, the portion of the distal outer sheath 40 which covers the stent 35 includes EAP material 232 in a spiral configuration. The EAP material 232 is connected to a lead 45 that extends proximally. When activated, the EAP material 232 causes an increase in the inside diameter of the distal outer sheath 40 from a first diameter, as shown in FIG. 11A, to a second diameter, as shown in FIG. 11B. This expansion breaks the striction forces between the stent 34 and distal outer sheath 40 and also reduces the force required for deployment of the stent 35. The activation of the EAP material 232 in the embodiment shown in FIG. 12 would function in a similar manner.

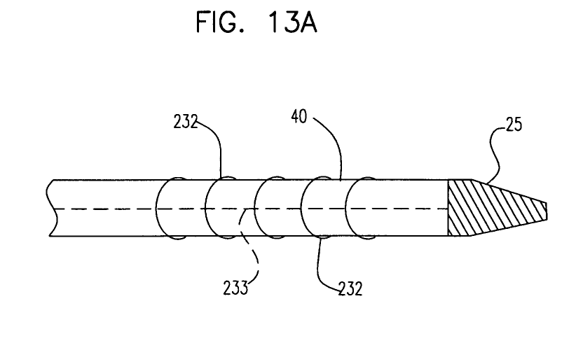

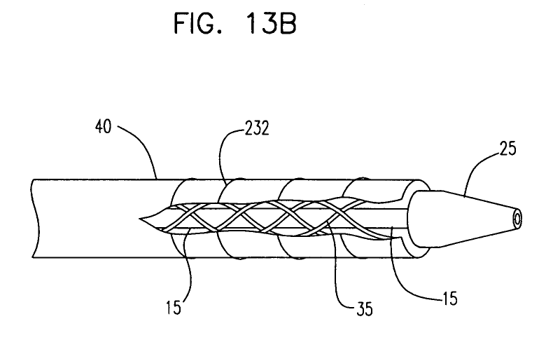

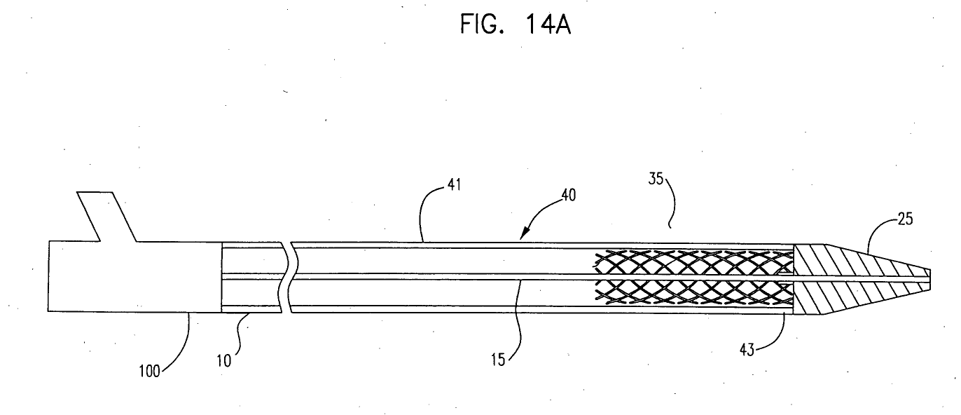

As can be seen in FIGS. 13A-B, the EAP material 232 may also be utilized to open the distal outer sheath 40 in a clamshell manner by forcing the distal outer sheath 40 to tear along a perforated or scored line 233. In this particular embodiment, the stent is about the guide wire shaft 15 or another such inner shaft and the EAP material 232 is shaped circumferentially such that there is a circumferential discontinuation of the EAP material 232 along a longitudinal line 233. Along this line 233, the distal outer sheath 40 has been perforated or scored. When activated, the EAP material 232 causes an increase in the diameter of the distal outer sheath 40 from a first diameter, as shown in FIG. 13A, tearing the distal outer sheath 40 along line 233, as shown in FIG. 13B. This tearing breaks the striction forces between the stent 34 and distal outer sheath 40 and also reduces the force required for deployment of the stent 35.

The manner of deployment of the stent 35 can be partial, as shown above in FIGS. 13A-B, or it could be utilized to fully deploy the stent. Full deployment could take place with a non-tubular stent, such as one rolled from a sheet, or from a tubular stent in a system where the inner does not pass through the center.

The distal outer sheath 40 pictured in FIGS. 13A and 13B could be used to reduce deployment forces for self-expanding stent delivery systems. In addition, 13B could be utilized to fully deploy a self-expanding stent and the delivery system is withdrawn thereafter. A method for deploying in this manner would be to locate the inner shaft 15 on one side of the tubular stent. Then when the outer sheath 40 is split the stent is free to deploy out of the split and the stent delivery system could then be withdrawn. The self-expanding stent 35 may be a self-expanding tube or may be an unwrapping sheet or coil.

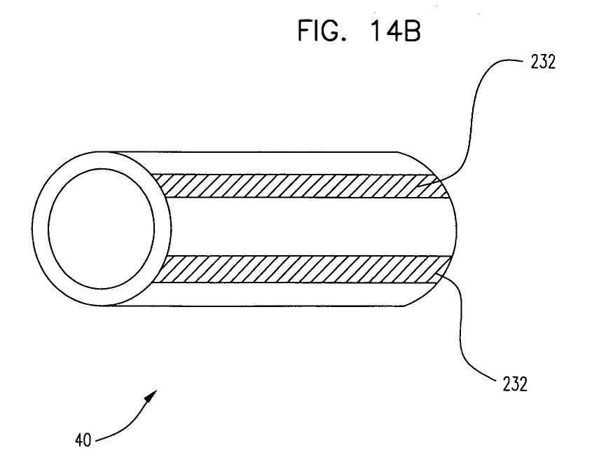

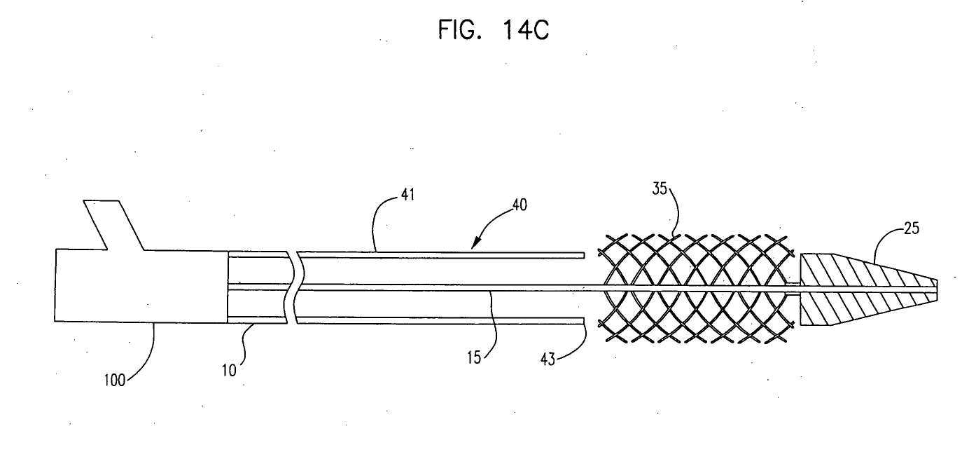

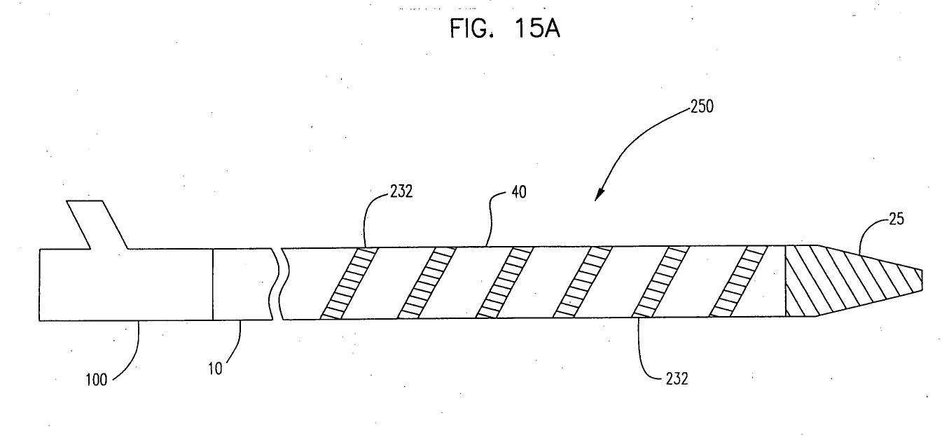

As shown in FIGS. 14A-C, EAP material 232 may be used on the entire distal outer sheath 40 or in longitudinal sections of the sheath 40, as shown in FIG. 14B. As mentioned above, the EAP material may be located on the outer surface, on the inner surface, inside the sheath 40 or comprise the entire wall thickness of the sheath 40. As current is applied, the entire sheath 40 shortens from a first position shown in FIG. 14A to a second position shown in FIG. 14C. Since the proximal end 41 of the distal outer sheath 40 is fixed on the manifold 100 or an optional proximal outer 10, the distal end 43 of the sheath 40 will retract, deploying the stent 35.

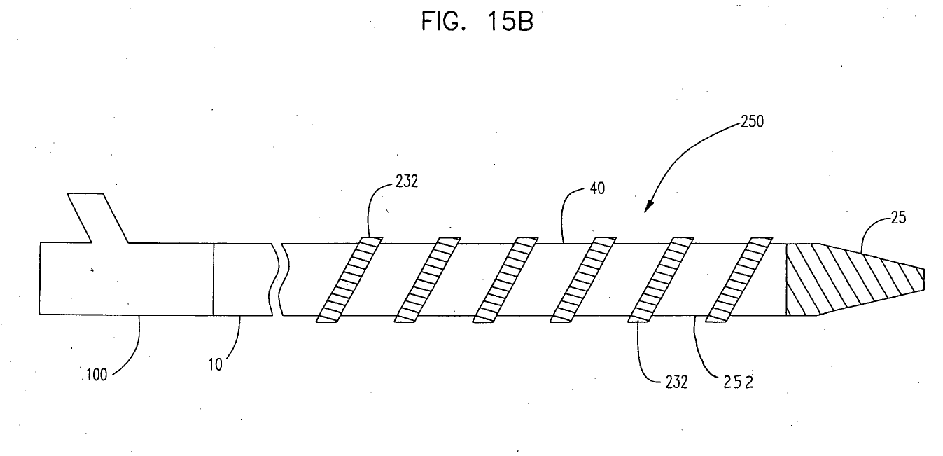

In at least one embodiment of the present invention, as shown in FIGS. 15A-B, a catheter 250 may have EAP material 232 on the outer surface of the distal outer sheath/shaft 40 and/or the proximal outer. In the figures shown, the EAP material 232 is just on the distal outer sheath/shaft 40. As shown in FIG. 15A, the EAP material 232 is in a spiral configuration along the distal outer sheath/shaft 40 and is substantially flush with the sheath/shaft 40. Upon activation, as shown in FIG. 15B, the stripes of EAP material 232 increase in radial thickness above the outer surface 252 of the distal outer sheath/shaft 40, thus increasing its profile. The activated EAP material 232 forms a propeller of sorts that can move fluid when the catheter is rotated. The profile may subsequently be reduced by deactivating the EAP material 232.

The present invention may be incorporated into both of the two basic types of catheters used in combination with a guide wire, commonly referred to as over-the-wire (OTW) catheters and rapid-exchange (RX) catheters. The construction and use of both over-the-wire and rapid-exchange catheters are well known in the art.

The present invention may also be incorporated into bifurcated assemblies. Examples of such systems are shown and described in U.S. patent application Ser. No. 10/375,689, filed Feb. 27, 2003 and U.S. patent application Ser. No. 10/657,472, filed Sep. 8, 2003 both of which are entitled Rotating Balloon Expandable Sheath Bifurcation Delivery; U.S. patent application Ser. No. 10/747,546, filed Dec. 29, 2003 and entitled Rotating Balloon Expandable Sheath Bifurcation Delivery System; U.S. patent application Ser. No. 10/757,646, filed Jan. 13, 2004 and entitled Bifurcated Stent Delivery System; and U.S. patent application Ser. No. 10/784,337, filed Feb. 23, 2004 and entitled Apparatus and Methodfor Crimping a Stent Assembly; the entire content of each of which are incorporated herein by reference.

Embodiments of the present invention can be incorporated into those shown and described in the various references cited above. Likewise, embodiments of the inventions shown and described therein can be incorporated herein.

In some embodiments the stent or other portion of the assembly may include one or more areas, bands, coatings, members, etc. that is (are) detectable by imaging modalities such as X-Ray, MRI or ultrasound. In some embodiments at least a portion of the stent, sheath and/or adjacent assembly is at least partially radiopaque.

A therapeutic agent may be placed on the stent 34 and/or the distal sheath 40, 75, in the form of a coating or by some other method such as the one shown in U.S. Pat. No. 6,562,065. Often the coating includes at least one therapeutic agent and at least one polymer. A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above materials throughout the application are intended for illustrative purposes only, and not as a limitation on the scope of the present invention. Suitable polymeric materials available for use are vast and are too numerous to be listed herein and are known to those of ordinary skill in the art.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

With this description, those skilled in the art may recognize other equivalents to the specific embodiment described herein. Such equivalents are intended to be encompassed by the claims attached hereto.

1. A catheter system comprising:
a catheter comprising a distal portion, a proximal portion and an inner shaft, the inner shaft comprising a medical device receiving region for receiving and carrying a medical device;
a distal sheath, wherein the distal sheath is about at least a portion of the medical device receiving region, the distal sheath having a first diameter, an inner surface and an outer surface and comprising at least one active region, wherein the at least one active region comprises electroactive polymers, wherein, upon stimulus to the at least one active region, the first diameter is expanded to a second diameter, wherein the second diameter is larger than the first diameter.

2. The catheter system of claim 1, further comprising a retraction mechanism in communication with the distal sheath, the retraction mechanism being capable of retracting the distal sheath from over the medical device receiving regions to allow for the release of the medical device from the catheter, wherein the distal sheath is expanded to its second diameter prior to retraction of the distal sheath.

3. The catheter system of claim 1, wherein the at least one active region is in the shape of a spiral.

4. The catheter system of claim 1, wherein the at least one active region is in the shape of a plurality of rings.

5. The catheter system of claim 4, wherein the plurality of rings are circumferentially discontinuous and wherein, upon expansion of the distal sheath to its second diameter, the distal sheath longitudinally tears exposing the medical device.

6. The catheter system of claim 1, wherein the at least one active region is on the inner surface of the distal sheath.

7. The catheter system of claim 1, wherein the electroactive polymer is an electric electroactive polymer or an ionic electroactive polymer.

8. The catheter system of claim 1, wherein the electroactive polymer is an electric electroactive polymer or an ionic electroactive polymer.

9. The catheter system of claim 8, wherein said electroactive polymer is an ionic electroactive polymer selected from the group consisting of conductive polymers, ionic polymer gels, ionomeric polymer-metal composites, carbon nanotubes and mixtures thereof.

10. The catheter system of claim 9, wherein said ionic electroactive polymer is a conductive polymer selected from the group consisting of polypyrroles, polyanilines, polythiophenes, polyethylenedioxythiophenes, poly(p-phenylene vinylene)s, polysulfones, polyacetylenes and mixtures thereof.

11. The catheter system of claim 2, the retraction mechanism comprising a pull back mechanism, wherein the pull back mechanism is controllable from the proximal region of the catheter for retraction of the distal sheath from over the medical device receiving region.

12. The catheter system of claim 11, wherein the pull back mechanism has a first length and comprises at least one active region, wherein the at least one active region comprises electroactive polymers, whereby, upon stimulus to the electroactive polymers, the first length is shortened to a second length, wherein the second length is shorter than the first length and wherein the shortening of the pull back mechanism causes the distal sheath to retract.

13. The catheter system of claim 12, wherein the pull back mechanism is a sheath oriented about the inner shaft.

14. A catheter system comprising:
a catheter comprising a distal portion, a proximal portion and an inner shaft, the inner shaft comprising a medical device receiving region for receiving and carrying a medical device;
a distal sheath, wherein the distal sheath is about at least a portion of the medical device receiving region, the distal sheath having a first diameter, an inner surface and an outer surface; and
a retraction mechanism in communication with the distal sheath, the retraction mechanism being capable of retracting the distal sheath from over the medical device receiving regions to allow for the release of the medical device from the catheter, wherein the retraction mechanism comprises at least one active region, the at least one active region comprising electroactive polymers, whereby, upon stimulus to the electroactive polymers, the retraction mechanism is shortened to a second length from a first length, wherein the second length is shorter than the first length and wherein the shortening of the retraction mechanism causes the distal sheath to retract.

15. The catheter system of claim 14, wherein the distal sheath comprises the retraction mechanism.

16. The catheter system of claim 15, wherein the retraction mechanism comprises longitudinal strips of EAP material.

17. The catheter system of claim 14, wherein the pull back mechanism is a sheath oriented about the inner shaft and wherein the pull back mechanism is proximal to the distal sheath.

18. The catheter system of claim 14, wherein the stimulus is electricity.

19. The catheter system of claim 14, wherein the electroactive polymer is an electric electroactive polymer or an ionic electroactive polymer.

20. The catheter system of claim 14, wherein the electroactive polymer is an electric electroactive polymer or an ionic electroactive polymer.

21. The catheter system of claim 20, wherein said electroactive polymer is an ionic electroactive polymer selected from the group consisting of conductive polymers, ionic polymer gels, ionomeric polymer-metal composites, carbon nanotubes and mixtures thereof.

22. The catheter system of claim 21, wherein said ionic electroactive polymer is a conductive polymer selected from the group consisting of polypyrroles, polyanilines, polythiophenes, polyethylenedioxythiophenes, poly(p-phenylene vinylene)s, polysulfones, polyacetylenes and mixtures thereof.

23. The catheter system of claim 14, the distal sheath further comprising at least one active region, wherein the at least one active region in the distal sheath comprises electroactive polymers, whereby, upon stimulus to the electroactive polymers, the first diameter is expanded to a second diameter, wherein the second diameter is larger than the first diameter, wherein the distal sheath is expanded to its second diameter prior to retraction of the distal sheath.

24. The catheter system of claim 23, wherein the at least one active region in the distal sheath is in the shape of a spiral.

25. The catheter system of claim 23, wherein the at least one active region in the distal sheath is in the shape of a plurality of rings.

26. The catheter system of claim 25, wherein the plurality of rings are circumferentially discontinuous and wherein, upon expansion of the distal sheath to its second diameter, the distal sheath longitudinally tears exposing the medical device.

27. The catheter system of claim 23, wherein the at least one active region on the distal sheath is on the inner surface of the distal sheath.

28. A catheter system comprising:
a catheter comprising a distal portion, a proximal portion and an inner shaft, the inner shaft;
a sheath disposed coaxially about the inner shaft, the sheath having an outer surface; and
EAP material, the EAP material being bonded to the outer surface of the sheath and comprising electroactive polymers, whereby, upon stimulus to the electroactive polymers, the EAP material radially expands from the surface of the sheath.

29. The catheter system of claim 28, wherein the EAP material is in the shape of a spiral around the sheath.

30. A catheter system comprising:
a distal shaft, a proximal shaft, and a midshaft disposed between and connected to the distal shaft and the proximal shaft, the midshaft having a first profile and comprising at least one active region, wherein the at least one active region comprises electroactive polymers, whereby, upon stimulus to the electroactive polymers, the first profile is reduced to a second and smaller profile.

31. The catheter system of claim 30, further comprising an inner shaft at least partially being disposed within the distal shaft.

32. The catheter system of claim 31, further comprising a port disposed between the midshaft and the distal sheath, wherein the port is in fluid communication with the inner shaft.
